(19) United States
(12) Patent Application Publication
(10) Pub. No.: US 2008/0119668 A1
(43) Pub. Date: May 22, 2008

(54) Method for the Extractive Purification of (Meth) Acrylic Acid by Using a Separating Agent

(76) Inventors: Stefan Nordhoff, Recklinghausen (DE); Axel Kobus, Langen (DE); Torsten Balduf, Pfungstadt (DE); Thomas Rathke, Mandeville, LA (US); Dennis Thong Yu-Chiang, Marl (DE); Jorg Leistner, Dortmund (DE)

Correspondence Address: SMITH MOORE LLP, P.O. BOX 21927, GREENSBORO, NC, 27420, US

(21) Appl. No.: 11/575,162

(22) PCT Filed: Sep. 13, 2005
(86) PCT No.: PCT/EP05/09836

§ 371 Date: Sep. 11, 2007

(30) Foreign Application Priority Data
Sep. 13, 2004 (DE) 10 2004 044 639.3

(51) Int. Cl. C07C 51/43; B01J 19/00

(52) U.S. Cl. 562/600; 422/187

(57) ABSTRACT

A process for the preparation of (meth)acrylic acid is disclosed that includes:
(a) contacting a product gas containing (meth)acrylic acid obtained from a gas phase oxidation with an aqueous phase in a quenching unit to obtain an aqueous quenched phase,
(b) contacting the quenched phase with an organic separating agent in an extraction unit to give a first phase and at least one other phase,
(c) crystallizing at least one of the first phase and the at least one other phase, such as by a suspension crystallization, in a crystallization unit to obtain a pure (meth)acrylic acid.

Also disclosed are a device for the preparation of (meth)acrylic acid, a process for the preparation of a polymer from the (meth)acrylic acid, a device for the preparation of the polymer, a polymer obtainable by this process, and the use of the (meth)acrylic acid or the polymer in a chemical product, such as fibers or shaped articles.

This application is a national stage application under 35 U.S.C. 371 of international application No. PCT/EP2005/009836 filed 13 Sep. 2005, and claims priority to German Application No. DE 10 2004 044 639.3 filed 13 Sep. 2004, the disclosure of which is expressly incorporated herein by reference.

The invention relates to a process for the preparation of (meth)acrylic acid, a device for the preparation of (meth)acrylic acid, a process for the preparation of a polymer from a (meth)acrylic acid produced by the above process, a device for the preparation of this polymer, a polymer produced by this process, and the use of the (meth)acrylic acid or the polymer in chemical products, such as fibers or shaped articles.

In the present context, (meth)acrylic acid is understood as meaning methacrylic acid and acrylic acid.

(Meth)acrylic acid, and in particular acrylic acid, is a monomer which is currently used in many polymers. In particular, acrylic acid is used in the preparation of polymers which are employed for water treatment, for example as flocculating agents, or which are incorporated as superabsorbent polymers into hygiene articles, in particular diapers (cf. Modern Superabsorbent Polymer Technology, F. L. Buchholz, A. T. Graham; Wiley-VCH 1998). When used both in the field of water treatment and in the field of hygiene articles, very high purity requirements are expected of the polymers employed there. This is the case in particular with hygiene articles, such as diapers, in which the polymer comes into contact with skin, directly or indirectly. This skin is very sensitive to contaminants, especially if they are irritating or toxic.

It is furthermore generally known that acrylic acid and methacrylic acid can be prepared by heterogeneous catalyzed gas phase oxidation of propylene with oxygen on catalysts which are in general in the solid state of aggregation, at temperatures of between 200 and 400° C. See DE OS 19 62 431, DE OS 29 43 707 and to DE 108 38 845 A1.

It is also known from WO 03/051809 A1 to bring a product gas containing (meth)acrylic acid obtained from the gas phase oxidation of propylene into contact with an aqueous phase to give an aqueous quenched phase. In such a quenched phase, the (meth)acrylic acid is still accompanied by various other reaction products, which are regarded as impurities, and by water as an absorption agent. This makes it necessary to feed the quenched phase obtained above to at least one further purification process. This purification process is often a distillation. Any distillation method with which a person skilled in the art is familiar and that seems suitable can be used in this context.

Japanese Patent Specification 32417/1970 discloses a process for the purification of acrylic acid, wherein a product gas obtained by gas phase oxidation is first absorbed with an absorption agent, such as water, in a quenching unit, and the quenched phase is fed to an extraction column in which a water-containing extraction phase is produced, which is fed to a distillation unit for removal of the water, the bottom product of this distillation column being subjected to crystallization.

The disadvantage of distillative purification of the quenched phase or of a bottom product such as that described in Japanese Patent Specification 32417/1970, however, is that distillation is a separating process involving high exposure to heat, which, in particular, in the purification of compositions containing (meth)acrylic acid leads to a high loss in yield as a result of a polymerization of the (meth)acrylic acid during the distillation, with the formation of (meth)acrylic acid dimers or (meth)acrylic acid oligomers.

DE 198 38 845 A1 discloses a process for the purification of acrylic acid, which can be crystallized directly out of a mixture with solvents, it being possible for the crystals to be present, for example, as a crystal suspension. The solvents are high-boiling solvents, the boiling points of which can be at least 20° C. above the boiling point of acrylic acid.

In general, the present invention is based on the object of overcoming the disadvantages arising from the prior art.

An object according to the invention is to provide a process for the purification of (meth)acrylic acid which can be operated in an environmentally-friendly and economical manner.

Another object of the invention is to provide a process for the purification of (meth)acrylic acid, in which the (meth)acrylic acid, which is known to be a reactive monomer, can be purified as carefully as possible. In this context, distillative purification steps should as far as possible be omitted, so that the loss of (meth)acrylic acid as a result of a formation of (meth)acrylic acid dimers or (meth)acrylic acid oligomers can be kept as low as possible.

Another object of the invention is to provide a process for the preparation of (meth)acrylic acid and for the purification thereof which can be carried out with as few steps as possible.

In addition, the invention is based on the object of providing products containing (meth)acrylic acid, which represent a low toxicological burden to users thereof.

The objects described above are achieved by the present invention.

The invention relates to a continuous process for the preparation of (meth)acrylic acid, wherein
(a) a product gas containing (meth)acrylic acid, obtained from a gas phase oxidation, is brought into contact with an aqueous phase in a quenching unit to give an aqueous quenched phase,
(b) the quenched phase is brought into contact with an organic separating agent in an extraction unit to give a first phase and at least one other phase,
(c) at least one phase chosen from the said first phase and the said one other phase, is subjected to a suspension crystallization or a layer crystallization in a crystallization unit to give pure (meth)acrylic acid.

By bringing the quenched phase into contact with the separating agent, the (meth)acrylic acid contained in the quenched phase is extracted from the quenched phase, forming two phases, wherein one phase is based predominantly on the separating agent and on (meth)acrylic acid, and the other phase can contain water and small amounts of (meth)acrylic acid. These phases can be fed directly to a crystallization device, in which the (meth)acrylic acid can crystallize in the form of a crystal suspension and then can be separated off by means of a washing column. Starting from the quenched phase obtained in a quenching unit up until a highly pure (meth)acrylic acid is obtained in a crystallization device, the process according to the invention thus requires no distillative separation step at all on any composition containing (meth)acrylic acid.

According to one embodiment of the present invention, the quenched phase can contain, as quenched phase components,
Q1 in the range of from 45 to 85 wt. %, in another aspect of an embodiment in the range of from 50 to 79 wt. %, and, in a further aspect of an embodiment, in the range of from 55 to 68 wt. % of (meth)acrylic acid,
Q2 at least 14.9 wt. %, in another aspect of an embodiment at least 20 wt. %, and in a further aspect of an embodiment at least 30 wt. % of water, and
Q3 at least 0.1 wt. %, in another aspect of an embodiment in the range of from 1 to 20 wt. %, in another aspect of an embodiment in the range of from 2 to 10 wt. %, and in a further aspect of an embodiment in the range of from 3 to 5 wt. % of an impurity other than Q1 and Q2, wherein each amount stated in each case is based on the total weight of the quenched phase,
   and wherein the sum of the wt. % stated for the quenched phase components Q1 to Q3 is 100.

In the extraction unit, the quenched phase is brought into contact with the separating agent in process step (b), the contact can take place at a temperature in the range of from −70 to +70° C., in another embodiment from −50 to +30° C. and in a further embodiment from −20 to +25° C.

By bringing the quenched phase into contact with the separating agent, a first phase and at least one other phase is obtained.

According to one embodiment of the claimed invention, the said first phase can contain
E1 10 to 70 wt. %, in another aspect of an embodiment 20 to 60 wt. %, and in a further aspect of an embodiment 30 to 50 wt. %, of (meth)acrylic acid,
E2 at least 29.9 wt. %, in another aspect of an embodiment 35 to 79.5 wt. %, and in a further aspect of an embodiment 40 to 49 wt. %, of separating agent as a main constituent of the first phase, and
E3 at least 0.1 wt. %, in another aspect of an embodiment 0.5 to 3 wt. %, and in a further aspect of an embodiment 1 to 2 wt. % of impurities other than E1 and E2, wherein each amount stated in each case is based on the total weight of the first phase,
   and wherein the sum of the wt. % states for components E1 to E3 is 100.
According to one embodiment of the claimed invention, the at least one other phase can be based on
W1 0.01 to 50 wt. %, in another aspect of an embodiment 0.5 to 20 wt. %, and in a further aspect of an embodiment 1 to 10 wt. % of (meth)acrylic acid,
W2 at least 49.9 wt. %, in another aspect of an embodiment 55 to 98.5 wt. %, and in a further aspect of an embodiment 80 to 96 wt. %, of water as a main constituent and
W3 at least 0.1 wt. %, in another aspect of an embodiment 1 to 10 wt. %, and in a further aspect of an embodiment 3 to 6 wt. %, of impurities other than W1 and W2, wherein each amount stated in each case is based on the total weight of the at least one other phase,
   and wherein the sum of the wt. % stated for components W1 to W3 is 100.

Low wt. % contents in the range of from 0.01 to 1 for W1 have proved suitable in a multi-stage procedure.

In principle, all the substances known to person skilled in a art are possible as the separating agent, and in one embodiment the separating agent can be in the form of a liquid at 100° C., in another aspect of an embodiment at 20° C., and under atmospheric pressure. Organic separating agents have proved suitable in the process according to the invention. These can be hydrocarbons, halogenated hydrocarbons, carbonyl compounds, alcohols, carboxylic acids, carboxylic acid esters, ethers, polyethers and an organic sulphur or phosphorus compound.

In one embodiment of the claimed invention, the separating agent can be a compound which is in the form of a liquid at 100° C., in another aspect of an embodiment at 20° C., and under atmospheric pressure, which leads to a two-phase system on contact with water in the weight ratio of 1:1. The separating agent can also be immiscible with water. According to one embodiment of the process of the present invention, a separating agent is employed of which no more than 1 g, in another aspect of an embodiment no more than 0.5 g, in a further aspect of an embodiment no more than 0.1 g, and yet in another aspect of an embodiment no more than 0.05 g, dissolves in one liter of water at 20° C.

Separating agents which can be used according to the invention and which can be employed by themselves or in mixtures of at least two are chosen from the group including
a hydrocarbon, such as, for example, n-hexane, n-heptane, dimethylcyclohexane, ethylcyclohexane, aromatics with an alkyl group, such as, for example, toluene, xylene or ethylbenzene, halogenated hydrocarbons, in particular halogenated aromatics, such as, for example, chlorobenzene, or mixtures of the above-mentioned hydrocarbons,
a carbonyl compound, such as, for example, acetone, acetaldehyde, diethyl ketone, diisopropyl ketone, methyl propyl ketone, methyl isobutyl ketone, methyl t-butyl ketone, n-nonanone or mixtures of these carbonyl compounds,
an alcohol or a polyol, such as, for example, methanol, n-propanol, iso-propanol, n-butanol, iso-butanol, tert-butanol, 1-decanol, glycerol or mixtures of these alcohols,
a carboxylic acid or a carboxylic acid ester, such as, for example, formic acid, acetic acid, n-propyl acetate, n-butyl acetate, ethyl acrylate, methyl methacrylate, ethyl methacrylate, vinyl acrylate, n-propyl acrylate, allyl acetate, isopropenyl acetate, vinyl propionate, propyl propionate, methyl crotonate, methyl valerate, ethyl butyrate or mixtures of these carboxylic acids or carboxylic acid esters,
an ether or a polyether, such as, for example, dimethyl ether, diethyl ether, ethyl methyl ether, dipropyl ether, methyl propyl ether, ethyl propyl ether, methyl propyl ether, ethyl methyl ether, dibutyl ether, ethylene glycol, propylene glycol, diethylene glycol dimethyl ether, diethylene glycol diethyl ether, triethylene glycol dimethyl ether and mixtures of these ethers or polyethers,
an organic sulphur, nitrogen or phosphorus compound, such as, for example, methanesulphonic acid, dioctylmonohexaphosphine (obtainable under the trade name Cyanex 923 from Cytec), trioctylphosphone oxide or mixtures of these compounds,
a solvent mixture containing a solvent A chosen from the group consisting of heptane, dimethylcyclohexane, ethylcyclohexane, toluene, benzene, ethylbenzene, chlorobenzene, xylene or a mixture of these solvents and a solvent B chosen from the group consisting of diethyl ketone, diisopropyl ketone, methyl propyl ketone, methyl isobutyl ketone, ethyl acrylate, methyl methacrylate, ethyl methacrylate, vinyl acrylate, n-propyl acrylate, allyl acetate, isopropenyl acetate, vinyl propionate, propyl propionate, methyl crotonate, methyl valerate, ethyl butyrate, dibutyl ether and mixtures of these solvents.

Ionic liquids can be separating agents in one embodiment of the invention. The term “ionic liquids” means compounds which have a melting point of no more than 150° C. in one aspect of an embodiment, of no more than 125° C. in another aspect of an embodiment, and no more than 100° C. in a further aspect of an embodiment, and a vapor at a temperature of 20° C., of no more than 1 mbar in one aspect of an embodiment, no more than 0.1 mbar in another aspect of an embodiment, of no more than 0.01 mbar in a further aspect of an embodiment, and of no more than 0.001 mbar in yet another aspect of an embodiment.

Examples of ionic liquids which can be employed according to the invention as separating agents are those compounds described as ionic liquids in WO 02/079269 A1. The disclosure of WO 02/079269 A1 concerning the ionic liquids is specifically incorporated by reference and represents a part of the disclosure of the present application. Examples of specific ionic liquids are trioctylmethylphosphonium hydrogen sulphate, trioctylmethylphosphonium methyl-sulphate, trioctylmethylammonium hydrogen sulphate and trioctylmethylammonium methyl-sulphate.

Separating agents which can be utilized in one embodiment of the present invention are aromatics in one aspect of an embodiment, being aromatics containing at least one alkyl group. Among these compounds, toluene, o-xylene, m-xylene, p-xylene, mesitylene, 1,4-trimethylbenzene, ethylbenzene or p-cymene or mixtures of at least two of these compounds can be utilized in another aspect of an embodiment, and toluene can be utilized as the separating agent in another aspect of an embodiment.

After the quenched phase in process step (a) has been brought into contact with the organic separating agent in the extraction unit to give a first phase and at least one other phase in process step (b), at least one of these two phases, the first phase in one aspect of an embodiment, optionally after this has been separated off from the other phase or phases, is fed in process step (c) to the crystallization, in one aspect of an embodiment the suspension crystallization or a layer crystallization, to give a pure (meth)acrylic acid.

In an embodiment of the process of the present invention, this one phase which is fed to the crystallization can be unpurified by means of distillation or rectification before carrying out the crystallization.

In another embodiment of the process according to the present invention, the mother liquor which is retained after the pure (meth)acrylic acid is obtained, and contains the separating agent, is at least partly recycled into the process according to the present invention, the at least partial recycling of the mother liquor can take place in one aspect of an embodiment after the quenching unit, and in another aspect of an embodiment at the end of the crystallization, and in a further aspect of an embodiment the suspension crystallization. In another aspect of an embodiment, the mother liquor can be recycled into the extraction unit. A very efficient use of the separating agent is ensured by this procedure. In this connection, in another aspect of an embodiment, the at least partial recycling of the mother liquor containing the separating agent can take place in that line via fresh separating agent (i.e., that which has not yet been brought into contact with the quenched phase), and which can be added after the quenching unit and at the latest during the crystallization in process step (c), is introduced.

In another embodiment of the process according to the invention, the recycling of the mother liquor can be carried out where it is first introduced through a separating unit (separator), which can be a crystallization unit, a layer crystallizer or a suspension crystallization unit comprising a crystal suspension generator and a separating device in another aspect of an embodiment, and in a further aspect of an embodiment, a washing column, or a centrifuge, a filter or another suitable device for separating off impurities, in order to first separate off impurities still present, in particular high-boiling components or low-boiling components, such as, for example, acetic acid. The mother liquor purified in this manner is then recycled into the process in the manner and method described above. In one embodiment of the process according to the invention, high-boiling components are separated off from the mother liquor by means of a separating unit, and in a further embodiment low-boiling components, such as, for example, acetic acid, are separated off. The use of at least two separation units arranged in series is also conceivable, so that first high-boiling components and then low-boiling components (or vice versa) can be separated off.

The other phase obtained in the extraction unit, which is substantially water-based, can be at least partly recycled into the quenching unit in one embodiment of the present invention. In another embodiment, this other phase can be subjected to a crystallization, in another aspect of an embodiment a suspension crystallization, in a purification device, for example a suspension crystallization unit or a layer crystallizer, before its recycling into the quenching unit, to give a pure (meth)acrylic acid and a mother liquor, the mother liquor obtained in this way, which is substantially water-based, then being fed to the quenching unit.

The first phase or the at least one other phase is separated off from the other phase or phases of the phase system in a procedure known to persons skilled in the art in conventional extraction processes. The separating off can be carried out with suitable separating devices, such as, for example, a separating funnel. Examples of the general procedure during the extraction can be found in Thornton J. D., “Science and Practice of Liquid-Liquid Extraction”, vol. I & II, Oxford, Oxford University Press, 1992, Lo T.-C., Baird M. H. I. and Hanson C., “Handbook of Solvent Extraction”, New York, John Wiley & Sons, 1983 and Robbins G. M. and Cusack R. W., “Liquid-Liquid Extraction Operations and Equipment” in “Perry's Chemical Engineering Handbook”, Perry R. H. and Green D. W., New York, McGraw Hill, chapter 15, 1997. The disclosures of these publications with respect to the general procedure for liquid-liquid extraction are hereby specifically incorporated herein by reference and represent a part of the disclosure of the present application.

In another embodiment, the process according to the present invention can be carried out continuously. In one aspect of an embodiment, the quenched phase obtained in process step (a) is fed continuously to an extraction unit and brought into contact there with the separating agent or with a mixture of separating agent recycled in accordance with the above disclosure and fresh separating agent which has not yet been brought into contact with the quenched phase. At least one of the two phases chosen from the first phase or the at least one other phase is removed from the extraction unit and fed to the crystallization unit in a continuous manner. The mother liquor which is retained in the crystallization unit and has a high content of separating agent is then at least partly recycled into the extraction unit, in one embodiment after prior purification.

The amount of fresh (i.e., not yet brought into contact with the quenched phase or with components of the quenched phase) separating agent employed in the process according to the claimed invention relative to the amount of the quenched phase depends on the extent to which separating agent which has already been brought into contact with the quenched phase is recycled in accordance with the above disclosure back into the process via the mother liquor which is retained in the crystallization unit and has a high content of separating agent. In this context, a person skilled in the art will be able to determine the amount of separating agent required for a satisfactory purification of the (meth)acrylic acid contained in the quenched phase as a function of the given process parameters (extent of recycling of the separating agent, composition of the quenched phase, nature of the extraction and crystallization processes used, reaction temperature, reaction pressure and the like) by routine experiments.

In one embodiment of the process according to the present invention, the amount of separating agent employed can be in a range of from 10 to 90 wt. %, in another aspect of an embodiment in a range of from 30 to 80 wt. %, and in a further aspect of an embodiment in a range of from 50 to 70 wt. %, based on the total weight of separating agent and quenched phase introduced into the process. In another embodiment, the weight ratio of freshly employed (i.e., not yet brought into contact with the quenched phase or components of the quenched phase) separating agent and separating agent recycled via the mother liquor retained in the crystallization unit can be in a range of from 1:1,000 to 1:100, in another aspect of an embodiment in a range of from 1:500 to 1:50, and in a further aspect of an embodiment in a range of from 1:200 to 1:100.

In the case of a continuously operated purification process, the separating agent can be added at various points. In one aspect of an embodiment, the separating agent can be added
into the feed line with which the quenched phase is led into the extraction unit,
into the extraction unit,
into the feed line with which the first phase obtained in the extraction unit is led into the crystallization unit,
into the crystallizer of the crystallization unit,
into the feed line with which, in the case of a suspension crystallization unit, the crystal suspension is led into the separating device, in one embodiment the washing column,
into the separating device, in one embodiment the washing column,
into the feed line with which the mother liquor retained in the crystallization unit is at least partly recycled into the extraction unit.

According to one embodiment of the process of the present invention, the pure (meth)acrylic acid obtained in process step (c) can contain at least 97.5, in another aspect of an embodiment at least 98.0, and in a further aspect of an embodiment at least 99.0, and in yet another aspect of an embodiment at least 99.5 wt. % of (meth)acrylic acid. Such units of (meth)acrylic acid may be utilized where a polymer which is prepared from the (meth)acrylic acid is used in the hygiene field, in the field of wound dressings and bandages or in other medical fields, such as medicament formulation or medical technology.

The invention furthermore relates to a device for the preparation of (meth)acrylic acid, comprising, connected to one another in series by fluid-carrying lines,
a reactor unit,
a quenching unit,
an extraction unit,
a first crystallization unit comprising a crystallizer, wherein the crystallization unit can be a layer crystallizer or a suspension crystallization unit which comprises, in addition to the crystallizer, a separating device, in one aspect of an embodiment a washing column, connected to the crystallizer, and optionally a further purification unit, in another aspect of an embodiment a crystallization unit comprising a crystallizer, wherein the further purification unit can be a layer crystallizer or a suspension crystallization unit,
   wherein
the quenching unit and the extraction unit, or
the extraction unit and the first crystallization unit or
the quenching unit and the extraction unit, and the extraction unit and the first crystallization unit are connected to one another without distillation.

In the context of the present invention, “connected to one another without distillation” means that the composition obtained in the quenching unit is not subjected to a purification step which comprises distillative separating off of one of the components of the composition, before it is fed into the extraction unit (the same applies accordingly to the connection without distillation between the extraction unit and the first crystallization unit). While as a rule the suspension crystallization unit is followed by a purification unit, in one aspect of an embodiment a washing column, this is, in general, not the case with the layer crystallizer.

According to the invention, the terms fluid-carrying means that the lines, in one embodiment pipelines, are constructed and configured such that these can carry gases or liquids or hypercritical fluids or solids as a slurry in liquids or at least two of these.

In connection with the embodiment of the reaction unit and the quenching unit, DE 198 38 845 A1 and WO 03/051809 A1, are hereby specifically incorporated herein by reference as part of this disclosure.

All the devices known to persons skilled in the art which allow separation into two liquid phases to be effected in a fluid and at least one, or in one aspect of an embodiment both, of these liquid phases to be removed from the extraction unit separately from one another, can be employed as the extraction unit. In one aspect of an embodiment, extraction devices are those which make it possible to carry out the process according to the invention continuously. In another aspect of an embodiment the extraction can take place by means of cross-current extraction, in which the quenched phase is mixed successively several times with fresh or recycled separating agent, or by means of counter-current extraction, in which the quenched phase and the separating agent or the recycled separating agent flow in counter-current through several extraction stages. Extractors which can be utilized include extraction batteries, in particular mixer/separator batteries, such as stirred extractors, circulating pump extractors, jet pump extractors, VENTURI tube extractors or ultrasound extractors, extraction columns with static inserts, such as, for example, packed columns, perforated tray columns or cascade columns, extraction columns with pulsation, such as, for example, pulsation columns (packed and perforated tray columns), extraction columns with moving inserts, such as, for example, stirred columns, rotating disc columns or whirler columns, and centrifugal extractors, such as, for example, the LUWESTA extractor, the QUADRONIC extractor, the PODBELNIAK extractor or the ALFA-LAVAL extractor.

In another embodiment of the present invention the extraction unit can be a temperature-controllable extraction unit, and in one aspect of an embodiment a cold extraction unit. These are employed in order to establish a temperature at which the mixture to be separated or purified has the widest possible miscibility gap.

Suspension crystallizers and layer crystallizers can be utilized in an embodiment of the present invention as the crystallizer.

Layer crystallizers which can be employed are those static and dynamic layer crystallizers which are mentioned as static or dynamic layer crystallizers in EP 0616 998 A1.

Suspension crystallizers which can be employed are all the devices suitable to the person skilled in the art with which crystal suspensions can be produced. Crystallizers which allow the process according to the invention to be carried out continuously can be utilized in one embodiment of the present invention. A stirred tank crystallizer, a scraped film crystallizer, a cooled disc crystallizer, a crystallizing screw, a drum crystallizer or the like can advantageously be arranged in the device according to the present invention as the crystallizer. This crystallizer can be the cooled disc crystallizer or the scraped film cooler (see Poschmann, Suspensionskristallisation organischer Schmelzen und Nachbehandlung der Kristalle durch Schwitzen oder Waschen [Suspension Crystallization of Organic Melts and After-treatment of the Crystals by Exudation or Washing], University of Bremen, Shaker Verlag, Aachen 1996). One embodiment of the device according to the present invention can comprise a scraped film cooler as the crystallizer. The suspension crystallization unit, moreover, has a separating unit for separating off the (meth)acrylic acid crystals, wherein this separating unit can be a washing column in which the mother liquor is stripped off from the column via a filter, resulting in the formation of a densely packed crystal bed. The mother liquor flows through the crystal bed in the direction of the base of the column and the bed is forced downwards by the flow resistance.

In another embodiment of the present invention the separating off of the (meth)acrylic acid by crystallization of the (meth)acrylic acid in the crystallization unit can be carried out in one stage or several stages, and in one or two stages in one aspect of the present invention. In the case of a two-stage crystallization, the mother liquor retained in the washing column or in the layer crystallizer after the (meth)acrylic acid crystals have been separated off is fed to a second crystallization unit. The second crystallization unit can be a layer crystallizer or a suspension crystallization unit. In the second crystallization unit, (meth)acrylic acid crystals are separated off to give a further mother liquor.

In one embodiment of the process according to the present invention, or of the device according to the present invention, the (meth)acrylic acid crystals separated off in the washing columns can be at least partly recycled into the suspension crystallization unit in which the crystal suspension fed to the respective washing column has been formed. The (meth)acrylic acid crystals can be at least partly melted before being recycled into the suspension crystallization unit, such as is described in DE 102 11 686 A1, which is specifically incorporated herein by reference. A particularly high purity of the (meth)acrylic acid can be achieved by this partial melting and recycling of the (meth)acrylic acid crystals. In the case where a washing column is used, this partial melting can be realized by locating a moving, in one aspect of an embodiment rotating scraping device or a scraper in the base of the column, which produces a suspension from the densely packed crystal bed and the wash melt introduced at the lower part of the washing column. The suspension can be pumped through a melter, which can be a heat exchanger in one aspect of the present invention, and melted. A part of the melt can serve, for example, as the wash melt. This is then pumped back into the column and in one embodiment of the present invention washes out the crystal bed migrating in the opposite direction, i.e. the crystallized (meth)acrylic acid is washed by the recycled (meth)acrylic acid in counter-current. On the one hand the wash melt effects washing of the crystals, and on the other hand the melt at least partly crystallizes out on the crystals. The crystallization enthalpy released heats the crystal bed in the washing region of the column. As a result, a purification effect analogous to exudation of the crystals is achieved.

In yet another embodiment of the present invention, at least a portion of the (meth)acrylic acid crystals obtained in the washing column can be fed into the suspension crystallization unit in crystalline form for seeding, such as is described in DE 102 11 686 A1.

In another embodiment of the device according to the present invention, in addition to the abovementioned components, a separating agent recycling, which starts from the first crystallization unit, in the case of a suspension crystallization unit which can be from the washing column of the first crystallization unit, and opens into the quenched phase line after the quenching unit or into the extraction unit. The mother liquor which is retained in the crystallization unit and has a high content of separating agent can be recycled into the extraction unit via this separating agent recycling.

In another embodiment of the present invention the device comprises a recycling for an aqueous phase from the extraction unit into the quenching unit. The at least one other phase, which contains water as the main constituent, can be recycled into the quenching unit via this recycling. In one embodiment this recycling can be interrupted by a further crystallization unit, which can be a layer crystallizer or a suspension crystallization unit comprising a crystallizer for the preparation of a crystal suspension in one aspect of an embodiment, and a washing column, those crystallizers and washing column which have already been mentioned above in connection with the first crystallization unit can be utilized in various embodiments.

In one embodiment of the process according to the present invention, and of the device according to the present invention, the first and/or second crystallization unit is a suspension crystallization unit comprising a crystallizer for the preparation of a crystal suspension and a separating device, in particular a washing column, connected to the crystallizer. In another embodiment of the process according to the present invention and of the device according to the present invention, the first and/or the second crystallization unit is a layer crystallizer.

The present invention also relates to the process described above for the preparation of (meth)acrylic acid, in which the device described above is employed.

The present invention also relates to a process for the preparation of a polymer, wherein a (meth)acrylic acid obtainable by the process described above is employed. In this context, the (meth)acrylic acid produced by the process of the present invention, such as the acrylic acid in one embodiment, is polymerized in the presence of free radical initiators and optional crosslinking agents, in a process of solution polymerization in one aspect of an embodiment, suspension polymerization or emulsion polymerization, optionally in the presence of further monomers which can be copolymerized with the (meth)acrylic acid obtainable by the process according to the invention and in the presence of crosslinking agents. The (meth)acrylic acid can be at least partly neutralized before, during or after the polymerization. In connection with the further monomers and crosslinking agents employed in the polymerization of the (meth)acrylic acid obtainable by the process according to the invention, reference is made to DE 101 61 495 A1, which is hereby specifically incorporated herein by reference as part of this disclosure, and there in particular to the monomers and crosslinking agents which are mentioned in this publication as monomers (α2) and as crosslinking agents (α3).

In connection with the process of the present invention for the preparation of a polymer, the present invention also relates to a device for the preparation of such a polymer, which comprises a device as described above, followed by a polymerization device.

The present invention also relates to a process for the preparation of a polymer which is at least partly based on (meth)acrylic acid, wherein the device described above is employed for the preparation of this polymer.

The present invention furthermore relates to fibers, films, foams and composites at least partly based on a (meth)acrylic acid produced by processes according to the present invention for the preparation of (meth)acrylic acid or containing the polymer according to the invention described above.

The present invention also relates to the use of a (meth)acrylic acid produced by the process according to the claimed invention for the preparation of (meth)acrylic acid or of a polymer produced by the process according to the claimed invention for the preparation of a polymer in or for the production of fibers, films, foams and composites.

The present invention will now be explained in more detail with the aid of non-limiting drawings and examples:

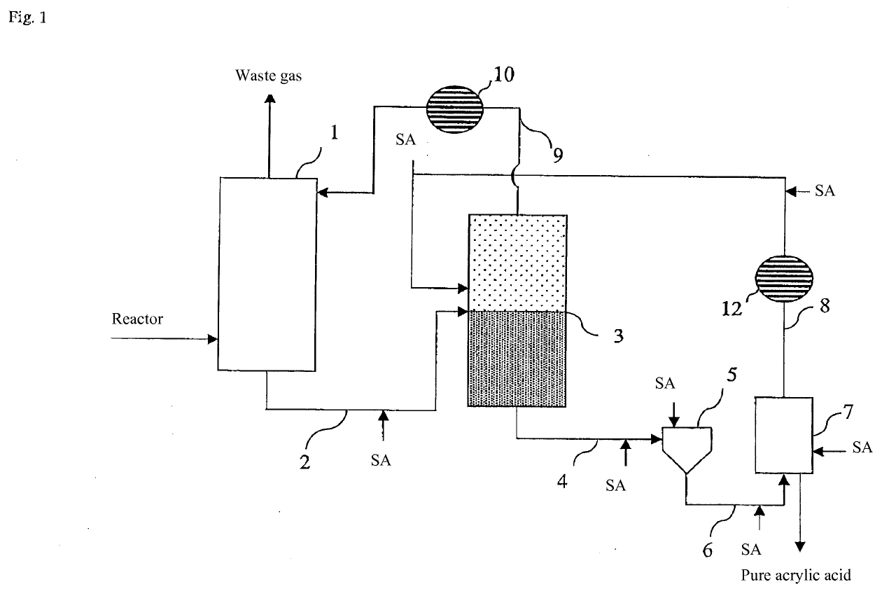

FIG. 1 shows the process according to the invention for the preparation of (meth)acrylic acid.

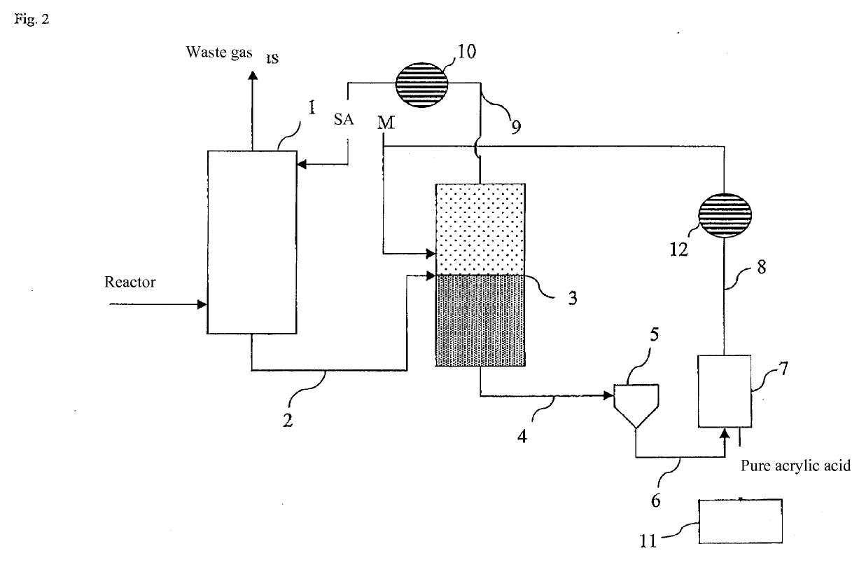

FIG. 2 shows the process according to the invention for the preparation of a polymer.

According to FIG. 1, the gaseous reaction product obtained in a reactor unit, which in the case of the oxidative gas phase oxidation of propylene comprises chiefly acrylic acid, water vapor, nitrogen, oxygen and by-products, such as, for example, maleic anhydride, is led into the quenching unit 1. There, by bringing into contact with a liquid, such as, for example, water, the gaseous reaction product is absorbed in the liquid, in the case where water is used as the absorption liquid an aqueous acrylic acid solution which also contains, in addition to (meth)acrylic acid and the absorption agent, the by-products obtained in the gas phase oxidation being obtained. This aqueous (meth)acrylic acid solution is passed via feed 2 into the extraction unit, it being brought into contact with the separating agent (SA) at the latest in the extraction unit. In the extraction unit, after the quenched phase has been brought into contact with the separating agent, a phase separation takes place, to form a first phase substantially based on the separating agent and at least one other phase substantially based on water. The phase substantially based on the separating agent is fed, in one embodiment without prior purification by distillation or rectification, via the feed 4 to the crystallizer 5, which can be a scraped film cooler. The crystal suspension obtained in the crystallizer 5 is then led via the feed 6 into the washing column 7, in which the (meth)acrylic acid crystals are separated off, in the course of which a mother liquor containing the separating agent is retained. The mother liquor obtained in the washing column 7 after the (meth)acrylic acid crystals have been separated off can be at least partly recycled via the feed 8 into the extraction unit 3. In another embodiment, the other phase obtained in the extraction unit 3, which is substantially based on water, can be recycled via the feed line 9 into the quenching unit 1. In one embodiment of the process according to the present invention the (meth)acrylic acid still contained in the composition led in the feed line 9 can be separated off from the composition by crystallization by means of a further purification device 10, which can be a suspension crystallization device or a layer crystallizer. In another embodiment of the process according to the invention, the composition introduced in the feed line 8 (mother liquor separated off during the crystallization) can be purified by means of a separating unit 12 before being recycled.

In the case of a continuously operated purification process, feeding of fresh separating agent (SA) can take place
a) into the feed 2 via which the quenched phase is introduced into the extraction unit,
b) into the extraction unit 3,
c) into the feed 4 with which the first phase obtained in the extraction unit, which contains the separating agent as the main constituent, is introduced into the crystallization unit,
d) into the crystallizer of the crystallization unit 5,
e) into the feed 6 with which the crystal suspension is introduced into the separating device 7,
f) into the separating device 7, or
g) into the feed 8 with which the mother liquor obtained in the separating device 7 is recycled into the extraction unit 3.

Feeding in at several of the abovementioned points of the purification process is also possible.

According to FIG. 2, the pure acrylic acid produced by the process according to the present invention is led into a polymerization device 11, in which a polymerization of the acrylic acid takes place, in one embodiment in aqueous solution, in the presence of a crosslinking agent and optional comonomers, to form a water-absorbent polymer.

### EXAMPLES

### Example 1

141 g of highly pure acrylic acid, 93 g of completely demineralized water and 73 g of toluene were initially introduced into a glass separating funnel temperature-controlled at 0° C. The mixture was shaken and left to stand for 30-50 minutes in order to allow a phase separation.

The two phases obtained were drained off separately, weighed and analyzed in respect of their composition (see the results in the following table). 104.2 g of upper phase and 202.5 g of lower phase were obtained.

The total amount of the upper phase was then transferred into a double-walled glass vessel and cooled to −30° C., while stirring. The crystallization started at −19.2° C. The crystal suspension formed was drained off and filtered over a vacuum filter. The compositions of the crystals and of the mother liquor are likewise shown in the following Table 1.

TABLE 1

| Fraction | Water (wt. %) | Acrylic acid (wt. %) | Toluene (wt. %) |
|---|---|---|---|
| lower phase | 44.3 | 52.6 | 3.1 |
| upper phase | 1.2 | 32.4 | 66.4 |
| mother liquor | 0.7 | 27.0 | 72.3 |
| crystals | 4.6 | 49.4 | 46.0 |

### Example 2

564 g of highly pure acrylic acid, 372 g of completely demineralized water and 292 g of toluene were initially introduced into a glass separating funnel temperature-controlled at 20° C. The mixture was shaken and left to stand for 180-240 minutes in order to allow a phase separation.

The two phases obtained were drained off separately, weighed and analyzed in respect of their composition (see the results in the following Table 2). 847.2 g of lower phase and 380.4 g of upper phase were obtained.

The upper phase was then transferred into a double-walled glass vessel and cooled to −30° C., while stirring. The crystallization started at −20.8° C. The crystal suspension formed was drained off and filtered over a vacuum filter. Half of the crystals obtained in this way were liquefied and cooled and used as the wash liquid for the remainder of the crystal fraction.

The compositions of the mother liquor, wash liquid and crystals are shown in the following Table 2.

TABLE 2

| Fraction | Water (wt. %) | Acrylic acid (wt. %) | Toluene (wt. %) |
|---|---|---|---|
| lower phase | 43.0 | 53.5 | 3.5 |
| upper phase | 1.2 | 30.5 | 68.3 |
| wash liquid | 5.2 | 60.3 | 34.4 |
| mother liquor | 0.3 | 25.8 | 73.9 |
| crystals | 5.2 | 90.5 | 4.3 |
| (washed) | | | |

### LIST OF REFERENCE SIGNS

1 Quenching unit
2 Feed for (meth)acrylic acid absorbed in the quenching unit into the extraction unit 3
3 Extraction unit
4 Feed for one of the liquid phases obtained in the extraction unit 3 into the crystallization unit comprising the crystallizer 5 and the washing column 7
5 Crystallizer (e.g. scraped film cooler)
6 Feed for crystal suspension into the washing column
7 Washing column
8 Feed for mother liquor into the extraction unit 3
9 Feed for one of the liquid phases obtained in the extraction unit 3 into the quenching unit 1
10 Optionally further purification device
11 Polymerization unit
12 Separating unit

1-24. (canceled)

25. A process for the preparation of (meth)acrylic acid, comprising the steps of:
(a) bringing into contact a product gas containing (meth)acrylic acid and obtained from a gas phase oxidation with an aqueous phase in a quenching unit to produce an aqueous quenched phase,
(b) bringing into contact the quenched phase with an organic separating agent in an extraction unit to produce a first phase and at least one other phase,
(c) subjecting to a crystallization in a crystallization unit at least one of the phases chosen from the first phase and the at least one other phase to produce a pure (meth)acrylic acid.

26. The process according to claim 25, wherein the quenched phase comprises:
Q1 about 45 to about 85 wt. % of (meth)acrylic acid,
Q2 at least about 14.9 wt. % of water, and
Q3 at least about 0.1 wt. % of impurities other than Q1 and Q2,
wherein the amounts stated in Q1 to Q3 are based on the total weight of the quenched phase, and wherein the sum of the wt. % stated for the quenched phase components Q1 to Q3 is 100.

27. The process according to claim 25, wherein the first phase comprises:
E1 about 10 to about 70 wt. % of (meth)acrylic acid,
E2 at least about 29.9 wt. % of separating agent as a main constituent of the first phase, and
E3 at least about 0.1 wt. % of impurities other than E1 and E2,
wherein the amounts stated in E1 to E3 are based on the total weight of the first phase, and wherein the sum of the wt. % stated for the quench phase components E1 to E3 is 100.

28. The process according to claim 25, wherein the other phase comprises:
W1 about 0.01 to about 50 wt. % of (meth)acrylic acid,
W2 at least about 49.9 wt. % of water as the main constituent of the other phase and
W3 at least about 0.1 wt. % of impurities other than W1 and W2,
wherein amounts stated in W1 to W3 are based on the total weight of the other the sum of the wt. % stated for quench phase components W1 to W3 is 100.

29. The process according to claim 25, wherein the crystallization is a suspension crystallization.

30. The process according to claim 25, further comprising the step of at least partly recycling the other phase into the quenching unit.

31. The process according to claim 25 further comprising the step of at least partly recycling the first phase into the extraction unit.

32. The process according to claim 25, wherein the separating agent is an aromatic comprising at least one alkyl group.

33. The process according to claim 32, wherein the aromatic comprising at least one alkyl group is chosen from the group of toluene, o-xylene, m-xylene, p-xylene, mesitylene, 1,2,4-trimethylbenzene, ethylbenzene or p-cumene or mixtures of at least two of these.

34. The process according to claim 25, wherein the bringing into contact in the extraction unit in step (b) takes place at a temperature in the range of from about −70 to about +70° C.

35. A device for the preparation of (meth)acrylic acid comprising:
a reactor unit,
a quenching unit,
an extraction unit,
a first crystallization unit comprising a crystallizer,
wherein the forgoing units are connected to one another in series by fluid-carrying lines; and
wherein:
the quenching unit and the extraction unit, or
the extraction unit and the first crystallization unit, or
the quenching unit and the extraction unit, and the extraction unit and the first crystallization unit,
are connected to one another without distillation.

36. The device according to claim 35 wherein the first crystallization unit comprises a further purification unit.

37. The device according to claim 36 wherein the further purification unit is a second crystallization unit.

38. The device according to claim 35, wherein the extraction unit is a temperature-controllable extraction unit.

39. The device according to claim 35, wherein a separating agent recycling starting from the first crystallization unit opens into a quenched phase feed after the quenching unit or into the extraction unit.

40. The device according to claim 35, wherein an aqueous phase recycling from the extraction unit into the quenching unit is provided.

41. The device according to claim 40, wherein the aqueous phase recycling is interrupted by a further purification unit.

42. The device according to claim 41 wherein the further purification unit is a second crystallization unit.

43. The device according to claim 42, wherein the first and second crystallization units are a suspension crystallization unit.

44. The device according to claim 43, wherein the suspension crystallization unit comprises a crystallizer and a separating device connected to the crystallizer.

45. A process for the preparation of a polymer, wherein a (meth)acrylic acid obtainable by a process according to claim 25 is polymerized.

46. A device for the preparation of a polymer, comprising a device according to claim 25 followed by a polymerization device.

47. A process for the preparation of a polymer at least partly based on (meth)acrylic acid, wherein a device according to claim 46 is employed.

48. Fibers, films, foams and composites, at least partly based on a (meth)acrylic acid obtainable by a process according to claim 25.

49. Use of a (meth)acrylic acid obtainable by a process according to claim 25 in or for the production of fibers, films, foams and composites.
